(19) United States
(12) Patent Application Publication
(10) Pub. No.: US 2024/0366817 A1
(43) Pub. Date: Nov. 7, 2024

(54) Alpha-emitter sources

(71) Applicant: Alpha Tau Medical Ltd., Jerusalem (IL)

(72) Inventors: Itzhak Kelson, Tel Aviv (IL); Michael Schmidt, Kfar Saba (IL); Ofer Magen, Hod Hasharon (IL); Dor Levy, Petach Tikva (IL); Ronen Segal, Jerusalem (IL); Amnon Gat, Matan (IL); Shai Lax, Givat Zeev (IL)

(21) Appl. No.: 18/648,395

(22) Filed: Apr. 28, 2024

Related U.S. Application Data
(60) Provisional application No. 63/499,268, filed on May 1, 2023.

(51) Int. Cl. A61K 51/12; A61K 9/00; A61K 51/02; A61K 51/06; A61N 5/10

(52) U.S. Cl. CPC A61K 51/1282; A61K 9/0024; A61K 51/025; A61K 51/06; A61K 51/1241; A61N 5/1014; A61N 2005/1024

(57) ABSTRACT

A radiotherapy source for treating a tumor, which includes a flexible core impermeable to radon and a polymer coating on the flexible core. The polymer coating is impermeable to radium, and allows diffusion of radon therethrough. The radiotherapy source also includes alpha-emitting radium radionuclides in the polymer coating or between the flexible core and the polymer coating.

### CROSS-REFERENCE TO RELATED APPLICATIONS

This application claims the benefit of U.S. provisional application 63/499,268, filed May 1, 2023, which is incorporated herein by reference in its entirety.

### FIELD OF THE INVENTION

The present invention relates generally to tumor therapy and particularly to intra-tumoral alpha-emitter radiation therapy.

### BACKGROUND OF THE INVENTION

Ionizing radiation is commonly used in the treatment of certain types of tumors, including malignant cancerous tumors, to destroy their cells. Alpha radiation is the most powerful radiation for cell destruction, buts its range is very short and therefore its delivery to tumors is a challenge.

Diffusing alpha-emitters radiation therapy (DaRT), described for example in U.S. Pat. No. 8,834,837 to Kelson, mounts alpha-emitting radionuclides on a source (also referred to as a seed) in a manner that the radionuclides do not leave the source, as a large percentage of the alpha-emitting radionuclides that leave the source are washed away through the blood stream before radioactive decay. On the other hand, the alpha-emitting radionuclides are mounted on the source in a manner such that a substantial percentage of their daughter radionuclides (radon-220 in the case of radium-224 and radon-219 in the case of radium-223) leave the source into the tumor, upon decay. These daughter radionuclides, and their own radioactive daughter atoms, spread around the source by diffusion up to a radial distance of a few millimeters before they decay by alpha emission. Thus, the range of destruction in the tumor is increased relative to radionuclides which remain with their daughters on the source.

PCT publication WO/2018/207105 titled: “Polymer Coatings for Brachytherapy devices” describes a DaRT source having a relatively thick polymer coating, which allows diffusion of daughter radionuclides therethrough.

Another method used to deliver alpha emitting radioactive atoms to malignant cells is targeted radionuclide therapy using radioimmunoconjugates. In targeted therapy, carriers, such as liposomes, are connected to radioactive atoms and injected into the blood stream of a patient. During circulation, the liposomes attach to malignant cells and when alpha particles are emitted by the radioactive atoms at least some of the emitted alpha particles destroy the malignant cells.

PCT publication WO01/60417 to Larsen, titled “Radioactive Therapeutic Liposomes”, PCT publication WO 02/05859 to Larsen, titled: “Method of Radiotherapy” and US patent publication 2004/0208821 to Larsen, titled: “Method of Radiotherapy”, the disclosures of which are incorporated herein by reference in their entirety, describe liposomes which encapsulate heavy radionuclides which emit alpha particles. The radionuclides may include, among others, Radium-223, Radium-224 and Thorium-227. Daughter radionuclides generally remain trapped during nuclear transformation of the radionuclides.

PCT publication WO2006/110889, titled: “Multi-Layer Structure having a Predetermined Layer Pattern Including an Agent”, describes a polymer multilayer structure which can be used to deliver radioisotopes for radiotherapy.

Alpha emitting radioactive atoms may also be delivered to a tumor in intracavitary treatment. US patent publication 2017/0000911, titled “Radiotherapeutic Particles and Suspensions”, describes intracavitary delivery of alpha-emitters coupled to microparticles or nanoparticles in a carrier, diluent or excipient. The microparticles and nanoparticles are described as either stable or slowly degrading.

PCT publication WO2010/028048, titled: “Brachytherapy Seed with Fast Dissolving Matrix for Optimal Delivery of radionuclides to Cancer Tissue”, describes polymer seeds embedding in them microspheres containing beta-emitting or alpha emitting radionuclides. After the seeds are implanted in a tumor the seeds are dissolved so that the radionuclides in the microspheres can destroy tumor cells.

U.S. Pat. No. 7,776,310 to Kaplan, titled “Flexible and/or elastic Brachytherapy Seed or Strand” describes a non-metal polymer brachytherapy flexible strand which carries alpha or beta emitting particles.

The paper Westrøm S, Malenge M, Jorstad I S, Napoli E, Bruland ØS, Bonsdorff T B, Larsen R H. Ra-224 labeling of calcium carbonate microparticles for internal α-therapy: Preparation, stability, and biodistribution in mice. J Labelled Comp Radiopharm. 2018 May 30; 61(6):472-486. doi: 10.1002/jlcr.3610. Epub 2018 Mar. 12. PMID: 29380410; PMCID: PMC6001669, proposes use of calcium carbonate microparticles as carriers for radium-224, designed for local therapy of disseminated cancers in cavitary regions.

### SUMMARY OF THE INVENTION

There is therefore provided in accordance with an embodiment of the present invention, a radiotherapy source for treating a tumor, comprising a flexible core impermeable to radon, a polymer coating on the flexible core, wherein the polymer coating is impermeable to radium, and allows diffusion of radon therethrough, and alpha-emitting radium radionuclides in the polymer coating or between the flexible core and the polymer coating.

Optionally, the flexible core comprises a gold strand. Alternatively or additionally, the flexible core comprises a polymer which is impermeable to radon. In some embodiments, the flexible core comprises Polyether ether ketone (PEEK). Optionally, the flexible core has a thickness not greater than 0.3 millimeters. Optionally, the radium radionuclides are dispersed throughout a thickness of the polymer coating. Optionally, the radiotherapy source further includes small particles dispersed in the polymer coating, wherein the radium radionuclides are coupled to the small particles. Optionally, the flexible core and the polymer coating are not biodegradable for at least a week from implantation in a tumor. Optionally, the radiotherapy source includes a layer of manganese oxide on the flexible core, and wherein the radium radionuclides are coupled to the layer of manganese oxide. Optionally, the radiotherapy source includes a layer of parylene or silicone rubber, between the flexible core and the layer of manganese oxide.

There is further provided in accordance with an embodiment of the present invention, a method for preparing a radiotherapy source, comprising mixing a solvent and solute to form a mixture which upon curing forms a polymer, mixing alpha-emitting radium radionuclides into the mixture, placing the mixture of radium and polymer components on a flexible core; and allowing the mixture to cure into a polymer coating, after placing the mixture on the flexible core. Optionally, mixing alpha-emitting radium radionuclides into the mixture comprises mixing a solution including radium radionuclides into the mixture. Optionally, the method includes removing excess liquid from the mixture, before placing the mixture on the flexible core.

There is further provided in accordance with an embodiment of the present invention, a medicament for treating a tumor, comprising microparticles having an outer surface comprising a manganese oxide; and alpha-emitter radium radionuclides on the outer surface of the microparticles. Optionally, the microparticles comprise a non-manganese-oxide core coated by manganese oxide. Optionally, the microparticles comprise gold, titanium, titanium oxide, zirconium oxide and/or silicon oxide. Optionally, the microparticles have a diameter of less than 10 micrometers.

### BRIEF DESCRIPTION OF THE DRAWINGS

FIG. 1 is a cross-section illustration of a flexible radiotherapy source, in accordance with an embodiment of the present invention;

FIG. 2 is a cross-section illustration of a flexible radiotherapy source, in accordance with another embodiment of the present invention;

FIG. 3 is a cross-section illustration of a flexible radiotherapy source, in accordance with still another embodiment of the present invention;

FIG. 4 is a flowchart of a method of production of flexible radiotherapy sources, in accordance with an embodiment of the present invention; and

FIG. 5 is a cross-section illustration of a directional radiotherapy source, in accordance with an embodiment of the present invention.

### DETAILED DESCRIPTION OF EMBODIMENTS

An aspect of some embodiments of the invention relates to a flexible radiotherapy source carrying alpha-emitter radium in a manner such that the radium is not released from the source, but daughter radon generated by radioactive decay of the radium is allowed to leave the flexible radiotherapy source. The flexible radiotherapy source comprises a flexible core which is impermeable to radon and an outer polymer coating which is permeable to radon but does not allow substantial passage of radium. Such flexible radiotherapy sources allow for the advantages of flexible sources, such as improved anchoring and matching to contours of a body organ, with diffusing alpha-emitters radiation therapy (DaRT).

An aspect of some embodiments of the invention relates to delivering alpha-emitter radium radionuclides to a tumor attached to small particles having an outer manganese oxide surface. Such small particles prevent release of radium from the tumor, while allowing daughter radon to diffuse into the tumor.

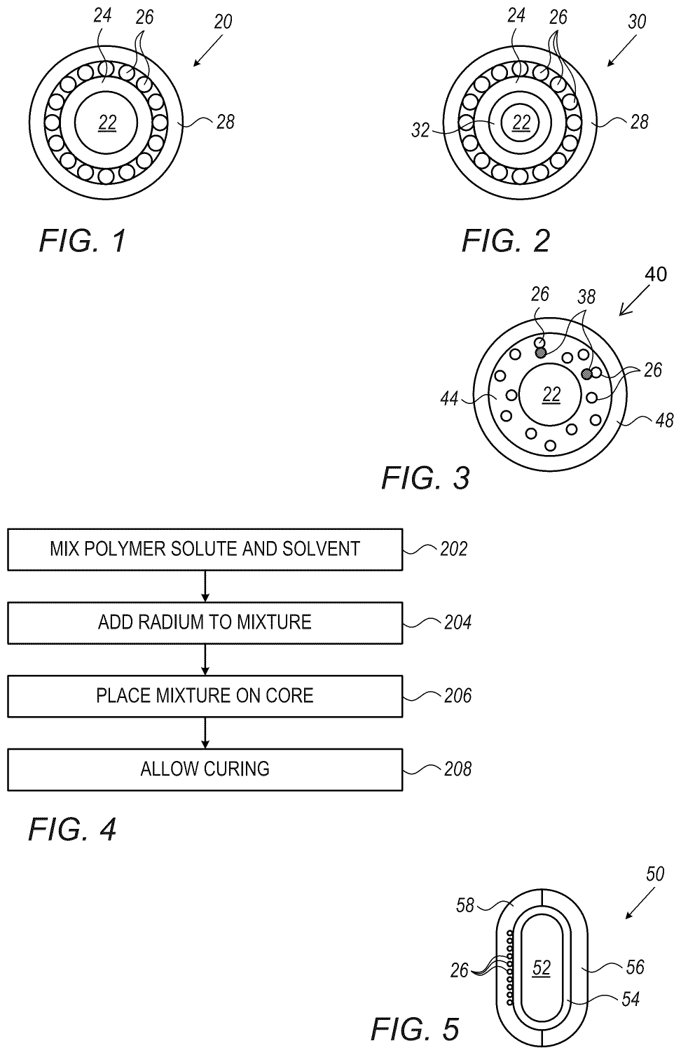

FIG. 1 is a cross-section illustration of a flexible radiotherapy source 20, in accordance with an embodiment of the present invention. Source 20 comprises an inner flexible core 22, a radium coupling layer 24, radium radionuclides 26 and a protective coating 28 which prevents leakage of radium and allows release of radon. It is noted that FIG. 1 and the other figures are not drawn to scale. Particularly, radium radionuclides 26 are drawn much larger than they actually are so that they can be seen in the figures.

In some embodiments, inner flexible core 22 comprises a flexible plastic metal, such as gold and/or titanium (e.g., pure titanium). In other embodiments, inner flexible core 22 comprises a flexible elastic metal such as nitinol. Inner flexible core 22 is optionally formed of a single thin mono-filament wire. Alternatively, inner flexible core 22 is braided from a plurality of thin wires.

In other embodiments, inner flexible core 22 comprises a polymer which is impermeable to radon, such as Polyether ether ketone (PEEK), Polytetrafluoroethylene (PTFE, sometimes known as Teflon), or polyimide (4,4′-oxydiphenylene-pyromellitimide, also known as Kapton).

Optionally, in embodiments in which inner flexible core 22 comprises a polymer, nanoparticles of a material which is easily identified in medical images, such as Gold, Platinum, Bismuth and/or Tantalum, are added to the polymer of inner flexible core 22 to allow identification of source 20 in medical images. The concentration of the nanoparticles may be low, for example, less than 10%, less than 5% or even less than 2%, but generally more than 0.5% or even more than 1%, or may be high, for example at least 30%, at least 50% or even more than 75%.

Inner flexible core 22 optionally has a cylindrical shape of a long and narrow strand, having a diameter of at least 0.1 millimeters, at least 0.15 millimeters or even at least 0.2 millimeters, to provide sufficient mechanical strength to the source. On the other hand, the diameter of inner flexible core 22 is optionally less than 0.5 millimeters, less than 0.4 millimeters, less than 0.35 millimeters or even less than 0.3 millimeters, so that the source is flexible. The diameter of inner flexible core 22 optionally depends on the material of inner flexible core 22. In one embodiment, inner flexible core 22 comprises a gold wire of a diameter of about 0.25 millimeters. Inner flexible core 22 is optionally solid without an internal channel, and thus differs from a tube with a hollow channel.

Flexible radiotherapy source 20 optionally has a bending moment of less than 5 Newton-millimeter (N*mm), less than 3 N*mm, less than 2 N*mm, less than 1 N*mm, less than 0.8 N*mm or even less than 0.6 N*mm. In some embodiments, flexible radiotherapy source 20 is bendable to a 90° deflection, within less than 2 centimeters or even not more than 1 centimeter.

In some embodiments, however, inner flexible core 22 has any other suitable shape, such as bars, balls, cylinders, pyramids, stars, and sheets, according to the intended purpose of the inner flexible core 22. In some embodiments, inner flexible core 22 is produced with a shape which matches a body region to be treated. For example, the shape of the body region is optionally determined using an imaging modality, such as CT, or by forming a mold on the body region, and inner flexible core 22 is produced with the determined shape using any suitable method known in the art, such as three-dimensional printing.

Radium coupling layer 24 optionally comprises manganese oxide. The manganese oxide optionally includes a hydrous manganese oxide (HMO) and/or manganese dioxide (MnO₂). Alternatively, the manganese oxide comprises any other manganese oxide which binds radium, such as manganese (IV) dioxide, manganese (II) oxide (MnO), manganese (II,III) oxide (Mn₃O₄), manganese (III) oxide (Mn₂O₃) and manganese (VII) oxide (Mn₂O₇) or a mixture of various manganese oxides.

Radium coupling layer 24 optionally has a thickness of not more than 1 micron, possibly less than 0.3 microns, less than 0.1 micron or even less than 0.05 microns. In some embodiments, radium coupling layer 24 has a thickness as low as about 10 nanometers.

The manganese oxide is optionally placed on inner flexible core 22 by dipping inner flexible core 22 in a potassium permanganate (KMnO₄) aqueous solution. The potassium permanganate (KMnO₄) is optionally at least 0.1%, at least 1% or even at least 3% of the aqueous solution by weight. Alternatively or additionally, the potassium permanganate (KMnO₄) is not more than 10% or even not more the 7% of the aqueous solution by weight. The dipping is optionally performed at a temperature of at least 60° Celsius, or even at least 80° Celsius, for example at about 90° Celsius. It is noted, however, that in some cases the dipping is performed at a temperature below 60° Celsius, for example at room temperature, or at temperatures higher than 90° Celsius, possibly as high as 150° Celsius. After dipping inner flexible core 22, inner flexible core 22 and the manganese oxide are optionally slowly cooled over at least an hour, or even at least 6 hours.

In other embodiments, radium coupling layer 24 comprises an agent which turns into a hydrogel by addition of calcium ions, such as sodium alginate or Pluronic. Optionally, in accordance with these embodiments, the agent which turns into a hydrogel is dried and solidified, for example by adding a high calcium percentage.

In still other embodiments, radium coupling layer 24 comprises a cation exchange resin, such as polystyrene treated by sulfonic acid, a copolymer of styrene and divinyl benzene treated by sulfonic acid and/or polyacrylate treated by carboxylic acid.

Optionally, radium radionuclides 26 comprise alpha-emitter radium-224 or radium-223. Radium radionuclides 26 are optionally placed on radium coupling layer 24, by dipping inner flexible core 22 with radium coupling layer 24 in a radium solution, for example as described in PCT publication WO2021/070029, which is incorporated herein by reference in its entirety. Alternatively, the radium radionuclides 26 are placed on radium coupling layer 24, by placing radium coupling layer 24 in a flux of radium radionuclides. The flux is optionally generated by a flux generating surface source. For example, when the radionuclide is Ra-224, a flux thereof can be generated by a surface source of thorium-228 (Th-228). A surface source of Th-228 can be prepared, for example, by collecting Th-228 atoms emitted from a parent surface source of U-232. Such parent surface source can be prepared, for example, by spreading a thin layer of acid containing U-232 on a metal. Alternatively or additionally, the flux is generated using any of the methods described in US patent publication 2015/0104560 to Kelson et al., titled: “Method and Device for Radiotherapy”, the disclosure of which is incorporated herein by reference in its entirety.

The thickness of protective coating 28 is optionally selected to allow radon created from desorption of radium radionuclides 26 to leave the source 20. Protective coating 28 is configured such that radium has a diffusion coefficient therein of less than 10⁻¹⁶ cm²/sec, less than 2*10⁻¹⁷ cm²/sec or even less than 10⁻¹⁸ cm/sec. On the other hand, radon optionally has a diffusion coefficient in protective coating 28, of at least 10⁻¹² cm²/sec, at least 10⁻¹¹ cm²/sec or even at least 10⁻¹⁰ cm²/sec.

Protective coating 28 is optionally non-biodegradable or at least does not degrade significantly (e.g., more than 0.1%) within five half-lives of radium radionuclides 26, from the time of implantation. It is noted, however, that in some embodiments, for example when the radium is coupled to microparticles and/or when the source carries a drug which is to be released, protective coating 28 and/or other parts of source 20 are biodegradable.

Protective coating 28 optionally comprises a polymer which is highly permeable to radon diffusion at body temperature, such as a silicone rubber (e.g., Polydimethylsiloxane (PDMS)) or polypropylene. Protective coating 28, when highly permeable may have a thickness of more than 10 microns, more than 20 microns or even more than 30 microns. Optionally, the thickness of highly permeable protective coating 28 is not more than 100 microns, is less than 70 microns is less than 50 microns or even is less than 20 microns. In some embodiments, the thickness of highly permeable protective coating 28 is less than 10 microns or even less than 5 microns.

The term impermeable to radon is used herein to refer to an element which does not allow passage of more than 1% of the radon which reaches the element. It is noted, however, that in some cases the material which is impermeable to radon does not allow passage of more than 0.1% or even of 0.01% of the radon which reaches the element.

Similarly, the term impermeable to radium is used herein to refer to an element which does not allow passage of more than 1% of the radium which reaches the element.

The term highly permeable to radon refers to an element which allows passage of at least 50% of the radon reaching the element. It is noted that in some cases, the element highly permeable to radon allows passage of at least 75%, or even at least 80% of the radon reaching the element. The term moderately permeable refers to an element which allows passage of between 10-50% of the radon reaching the element. An element which allows diffusion of radon is one which allows diffusion of at least 10% of the radon reaching the element. The element could be moderately permeable to radon or highly permeable. Alternatively to a polymer which is highly permeable, protective coating 28 comprises a polymer which is moderately permeable to radon diffusion at body temperature, such as polycarbonate, polyethylene terephthalate, poly(methyl methacrylate), polysulfone and/or parylene. In accordance with this alternative, the thickness of protective coating 28 is less than 10 microns, less than 6 microns or even less than 3 microns, so as to allow diffusion of radon therethrough and to allow simple measurement of the activity of radium radionuclides 26 on source 20. On the other hand, the thickness of protective coating 28 is optionally greater than 0.1 microns, greater than 0.25 microns, greater than 0.5 microns, greater than 1 micron or even greater than 2 microns to prevent radium escape.

The material and thickness of protective coating 28 are selected such that at least 20%, at least 35%, at least 40%, at least 45%, at least 60%, at least 80% or even at least 90% of the radon radionuclides resulting from desorption of radium radionuclides 26 leave source 20. The radon radionuclides leave protective coating 28 due to the desorption energy and/or at a later time due to diffusion. In stating that radium radionuclides 26 do not leave protective coating 28 it is intended that at most a negligible amount of the radium radionuclides 26, such as less than 1% or even less than 0.1% leave outer polymer layer 24 before desorption.

FIG. 2 is a cross-section illustration of a flexible radiotherapy source 30, in accordance with another embodiment of the present invention. Source 30 is similar to source 20 of FIG. 1, in that it comprises an inner flexible core 22, a radium coupling layer 24, radium radionuclides 26 and a protective coating 28 which prevents leakage of radium radionuclides 26 and allows release of radon. Source 30, however, additionally comprises an intermediary layer 32 between inner flexible core 22 and radium coupling layer 24 to strengthen their coupling. In some embodiments in which radium coupling layer 24 comprises manganese oxide, intermediary layer 32 comprises a layer of parylene or silicone rubber (e.g., Polydimethylsiloxane (PDMS)), which couples to the manganese oxide and strengthens its coupling to inner flexible core 22. Intermediary layer 32 has a thickness sufficient to form a bond both with inner flexible core 22 and with radium coupling layer 24, of at least 0.2 micron, at least 0.25, at least 0.4 microns or even at least 0.5 microns. Intermediary layer 32 optionally has a thickness of less than 10 microns, less than 6 microns, less than 4 microns, or even less than 3 microns, so as not to allow the material of radium coupling layer 24 to sink into intermediary layer 32 in a manner which will damage the coupling of radium radionuclides 26 to radium coupling layer 24.

Source 30 is particularly useful when inner flexible core 22 comprises a polymer and radium coupling layer 24 comprises manganese oxide, which do not couple well.

FIG. 3 is a cross-section illustration of a flexible radiotherapy source 40, in accordance with still another embodiment of the present invention.

Source 40 comprises inner flexible core 22 as discussed regarding source 20, and thereon includes an outer polymer layer 44, which carries in it radium radionuclides 26. In some embodiments, the structure of outer polymer layer 44 is such that radium radionuclides 26 do not leave outer polymer layer 44 to a significant extent (e.g., more than 1%, more than 3% or more than 5%) but a significant percentage of radon radionuclides resulting from desorption of the radium radionuclides 26 leave outer polymer layer 44. In other embodiments, source 40 additionally comprises a protective coating 48, which prevents leakage of radium radionuclides 26.

Inner flexible core 22 provides strength to source 20 to prevent tear and/or provides visibility of source 20 in one or more medical imaging modalities. The thickness of outer polymer layer 44 is optionally selected to allow radon created from radium desorption throughout the outer polymer layer 44 to leave the source 40.

Outer polymer layer 44 optionally comprises a polymer which is highly permeable to radon diffusion at body temperature, such as a silicone rubber (e.g., Polydimethylsiloxane (PDMS)) or polypropylene. Outer polymer layer 44 when highly permeable may have a thickness of more than 10 microns, more than 20 microns or even more than 30 microns. Optionally, the thickness of highly permeable outer polymer layer 44 is not more than 100 microns, is less than 70 microns is less than 50 microns or even is less than 20 microns. In some embodiments, the thickness of highly permeable outer polymer layer 44 is less than 10 microns or even less than 5 microns.

Alternatively to a polymer which is highly permeable, outer polymer layer 44 comprises a polymer which is moderately permeable to radon diffusion at body temperature, such as polycarbonate, polyethylene terephthalate, poly(methyl methacrylate), polysulfone and/or parylene. In accordance with this alternative, the thickness of outer polymer layer 44 is less than 10 microns, less than 6 microns or even less than 3 microns, so as to allow diffusion of radon therethrough and to allow simple measurement of the activity of radium radionuclides 26 on source 20.

The polymer forming outer polymer layer 44 is optionally non-biodegradable or at least does not degrade significantly (e.g., more than 0.1%) within five half-lives of the radium, from the time of implantation. It is noted, however, that in some embodiments, for example when the radium is coupled to microparticles and/or when the source carries a drug which is to be released, the source is biodegradable.

Outer polymer layer 44 is configured such that radium has a diffusion coefficient therein of less than 10⁻¹⁶ cm²/sec, less than 2*10⁻¹⁷ cm²/sec or even less than 10⁻¹⁸ cm²/sec. On the other hand, radon optionally has a diffusion coefficient in outer polymer layer 24, of at least 10⁻¹² cm²/sec, at least 10⁻¹¹ cm²/sec or even at least 10⁻¹⁰ cm²/sec.

Protective coating 48, when included in source 40, optionally has a thickness greater than 0.1 microns, greater than 0.25 microns, greater than 0.5 microns, greater than 1 micron or even greater than 2 microns to prevent radium escape. Preferably, the thickness of protective coating 48 is less than 5 microns, less than 4 microns, less than 3 microns or even less than 2 microns. Protective coating 48 comprises any of the materials discussed above regarding protective coating 28.

The material and thickness of outer polymer layer 44 are selected such that at least 20%, at least 35%, at least 40%, at least 45%, at least 75% or even at least 85% of the radon radionuclides resulting from desorption of the radium radionuclides leave outer polymer layer 44. The radon radionuclides leave outer polymer layer 44 due to the desorption energy and/or at a later time due to diffusion. In stating that radium radionuclides 26 do not leave outer polymer layer 44 it is intended that at most a negligible amount of the radium, such as less than 1% or even less than 0.1% leave outer polymer layer 44 before desorption.

Radium radionuclides 26 are optionally dispersed homogenously in outer polymer layer 44. Particularly, radium radionuclides 26 are optionally dispersed substantially evenly throughout the thickness of outer polymer layer 44. The holding of radium radionuclides 26 in polymer layer 44 removes the need for radium coupling layer 24, and therefore, in some embodiments, source 40 does not include radium coupling layer 24.

In some embodiments, radium radionuclides 26 are included in outer polymer layer 44 as free atoms which are not coupled to larger particles. In other embodiments, radium radionuclides 26 are coupled to small particles 38. In some embodiments, small particles 38 have a diameter of less than 100 micrometers, less than 50 micrometers, less than 10 micrometers or even less than 5 micrometers. Even smaller microparticles, such as nanoparticles, are used in some embodiments.

In the present application, the term “microparticles” refers to particles having a diameter of between 1 and 100 micrometers. The term “nanoparticles” refers to particles having a diameter of between 100 and 1000 nanometers. In some embodiments, the small particles are spheres and/or beads. Alternatively, the small particles are of any other suitable shape.

Optionally, the small particles 38 comprise a material which is easily identifiable using a medical imaging modality such as ultrasound, x-ray and/or magnetic resonance imaging (MRI). Alternatively or additionally, the small particles include within them markers of a material easily identifiable in medical images.

The small particles comprise, in some embodiments, gold, titanium, titanium oxide, aluminum oxide, zirconium oxide and/or silicon oxide, although other materials may also be used for the small particle bases.

In some embodiments, the small particles are coated by a thin layer of a manganese oxide which attracts radium. The thin layer of manganese oxide is optionally sufficiently thick to couple radium, for example of a thickness of at least 10 nanometers, at least 20 nanometers, at least 30 nanometers, at least 50 nanometers or even at least 80 nanometers. Optionally, the thin layer has a thickness of less than 10 microns, less than 1 micron, less than 500 nanometers, less than 250 nanometers, less than 150 nanometers or even less than 100 nanometers, so as to minimize the amount of manganese oxide injected to the patient. Alternatively, the small particles are mostly or even entirely made of manganese oxide. The manganese oxide coating of the small particles is sufficiently thin so that the radon is released from the small particle when created by decay of the radium radionuclides 26.

FIG. 4 is a flowchart of a method of production of flexible radiotherapy sources, in accordance with an embodiment of the present invention. The method includes mixing (202) a polymer solute which forms outer polymer layer 44 in a solvent, and adding (204) radium radionuclides 26 to the mixture. The mixture is then placed (206) on inner flexible core 22 and is allowed to cure (208).

Exemplary solutions and solvents which may be used are listed in Table 1.

TABLE 1

| Solution | Solute | Solvent |
|---|---|---|
| 1 | Polypropylene | Xylene (e.g., heated |
| | | to ~100 C.) |
| 2 | Polycarbonate | Dichloromethane (DCM) |
| 3 | Polydimethylsiloxane (PDMS) | Hexane (before curing) |
| 4 | Polyethylene terephthalate (PET) | Hexafluoroisopropanol (HFIP) |
| 5 | Poly(methyl methacrylate) | DCM |
| 6 | Polysulfone | DCM |

The solutes and solvents are presented solely by way of example and it will be understood that any other suitable combinations of solutes and solvents may be used. The mixing of the solvent and solute is performed using methods known in the art. As is known in the art, for some materials, such as polypropylene, the mixing is performed at high temperatures required for extrusion, while for other materials the mixing is performed at room temperature.

In some embodiments, radium radionuclides 26 are acquired in an aqueous acidic solution, having a mild acidity, for example of pH above 3.5, above 4 or even above 4.5. Alternatively, radium radionuclides 26 are acquired in a neutral solution, such as potassium chloride (KCL).

In other embodiments, radium radionuclides 26 are provided combined to small particles 38, optionally as a dry powder. The radium radionuclides 26 are optionally coupled to the small particles by placing the small particles in a flux of radium radionuclides. The flux is optionally generated by a flux generating surface source. For example, when the radionuclide is Ra-224, a flux thereof can be generated by a surface source of thorium-228 (Th-228). A surface source of Th-228 can be prepared, for example, by collecting Th-228 atoms emitted from a parent surface source of U-232. Such parent surface source can be prepared, for example, by spreading a thin layer of acid containing U-232 on a metal. Alternatively or additionally, the flux is generated using any of the methods described in US patent publication 2015/0104560 to Kelson et al., titled: “Method and Device for Radiotherapy”, the disclosure of which is incorporated herein by reference in its entirety.

Alternatively, alpha-emitter radium radionuclides 26 are coupled to the small particles 38 by mixing the radium and small particles in an aqueous solution. A radium solution can be generated by dissolving seeds carrying radium radionuclides into a solution. Further alternatively or additionally, a high concentration solution including the alpha-emitter radium radionuclides is generated, for example using any of the methods described in PCT publication 2021/070029, titled “Wet preparation of radiotherapy sources”, the disclosure of which is incorporated herein by reference in its entirety. The small particles, with their radium-attracting outer coating are optionally immersed in the high concentration solution for several hours in order to coat the small particles 38 with radium radionuclides 26. The solution may be added (204) to the polymer mixture or may be dried and then the remaining dry small particles 38 carrying the radium radionuclides 26 are mixed with the polymer mixture. The small particles 38 are optionally sufficiently large (e.g., having a largest dimension of at least 0.1 micrometers, at least 1 micrometer or even at least 5 micrometers), in these embodiments, to allow handling of the radium without the solution as a carrier.

As to adding (204) the radium radionuclides 26 to the polymer, when radium radionuclides 26 are in an aqueous solution, after adding (204) the radium solution to the mixture (e.g., a mixture of PDMS components), the mixture is optionally emulsified and then excess liquid is optionally removed by vacuum suction, to allow proper curing of the polymer. Optionally, before mixing the polymer components with the radium solution, the polymer component mixture is diluted by a water repellant (e.g., Hexane). Then, the radium solution and polymer components are mixed together and the excess liquids are removed.

In embodiments in which the radium is provided as a dry powder, the powder is mixed directly into the mixed components of the polymer and the removal of liquids is not required.

In some embodiments, the polymer mixture with radium radionuclides 26 mixed therein is placed (206) on inner flexible core 22, by dipping inner flexible core 22 in the mixture. Alternatively, the polymer mixture is mixed in a syringe and when the polymer components and the radium are properly mixed the contents is pushed out of a small nozzle of the syringe having a cross-section of a desired cross-section of the produced source. Inner flexible core 22 is placed at the exit of the nozzle and the polymer mixture exiting the nozzle wraps around inner flexible core 22.

FIG. 5 is a cross-section illustration of a directional radiotherapy source 50, in accordance with an embodiment of the present invention. Source 50, as shown, differs from source 20 in that it is elongated rather than round. This difference illustrates what was already stated above, that the sources of the present invention may be in various shapes.

Source 50 further differs from source 20 in that it is designed to emit radon in a specific direction, rather than in all directions. Source 50 includes an inner flexible core 52, of any of the materials discussed above regarding inner flexible core 22 and a thickness similar to the diameter of inner flexible core 22. Source 50 further comprises a radium coupling layer 54 similar to radium coupling layer 24, radium radionuclides 26 and a protective coating 58, of materials and thickness similar to protective coating 28. However, radium coupling layer 54, radium radionuclides 26 and protective coating 58 cover only part of the perimeter of inner flexible core 52, such that daughter radionuclides are released only in those directions. In some embodiments, the remaining parts of the circumference of core 52 are covered by a neutral coating 56 which does not carry radium. Neutral coating 56 optionally comprises a material which is impermeable to radon, such as any of the materials discussed above forming core 22. Source 50 can be used on the outer periphery of a tumor, when it is desired to release radium only in the direction of the tumor.

Alternatively, radium coupling layer 54 and radium radionuclides 26 are placed on the entire perimeter of inner flexible core 52, and protective coating 58 is made thicker, for example of at least 100 microns, or even at least 200 microns, in directions in which radiation is undesired.

In some embodiments, in addition to alpha-emitter radium radionuclides 26, the sources 20, 30, 40 and/or 50 include one or more drugs to be administered in parallel to the alpha-emitter radiation treatment. The one or more drugs optionally include a substance which activates cytoplasmatic sensors for intracellular pathogen in the tumor, as described for example in PCT publication WO 2020/089819, titled: “Intratumoral Alpha-Emitter Radiation and Activation of Cytoplasmatic Sensors for Intracellular Pathogen”, the disclosure of which is incorporated herein by reference in its entirety. Alternatively or additionally, the one or more drugs include an immune checkpoint regulator, such as described in PCT application PCT/IB2022/055680, titled: “Intratumoral Alpha-Emitter Radiation in combination with Checkpoint Regulators”, the disclosure of which is incorporated herein by reference in its entirety. Further alternatively or additionally, the one or more drugs include a vasculature inhibitor, such as described in PCT application PCT/IB2022/055679, titled: “Intratumoral Alpha-Emitter Radiation in combination with Vasculature Inhibitors”, the disclosure of which is incorporated herein by reference in its entirety. In some embodiments, the one or more drugs are included in the small particles. Alternatively or additionally, the one or more drugs are placed in or on outer polymer layer 24.

Sources 20, 30, 40 and 50 are particularly useful for tumors of cancer types in delicate organs, in which it is preferred to implant soft sources, such as in the brain and/or lungs. In some embodiments, sources 20, 30, 40 and/or 50 are implanted in a tumor in an orientation with multiple bends, thus better anchoring the source in the tumor.

Small particles with a manganese coating which carry radium may be used for treating cancer also in sources other than those described above. The manganese-coated small particles may be delivered on their own, in a solution, in a gel or in any other suitable method. Gels which may be used are listed in Xian Jun Loh, “In-Situ Gelling Polymers, for Biomedical Applications”, 2014, the disclosure of which is incorporated herein by reference.

Alternatively to using manganese-coated small particles, radium is provided in a solution carrying manganese, or any other suitable radium coupler, in a raw form. For example, the manganese, or other suitable radium coupler is optionally provided as a powder, which attracts radium.

It will be appreciated that the above described methods and apparatus are to be interpreted as including apparatus for carrying out the methods and methods of using the apparatus. It should be understood that features and/or steps described with respect to one embodiment may sometimes be used with other embodiments and that not all embodiments of the invention have all of the features and/or steps shown in a particular figure or described with respect to one of the specific embodiments. Tasks are not necessarily performed in the exact order described.

It is noted that some of the above described embodiments may include structure, acts or details of structures and acts that may not be essential to the invention and which are described as examples. Structure and acts described herein are replaceable by equivalents which perform the same function, even if the structure or acts are different, as known in the art. The embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art. Therefore, the scope of the invention is limited only by the elements and limitations as used in the claims, wherein the terms “comprise.” “include.” “have” and their conjugates, shall mean, when used in the claims. “including but not necessarily limited to.”

1. A radiotherapy source for treating a tumor, comprising:
a flexible core impermeable to radon;
a polymer coating on the flexible core, wherein the polymer coating is impermeable to radium, and allows diffusion of radon therethrough; and
alpha-emitting radium radionuclides in the polymer coating or between the flexible core and the polymer coating.

2. The radiotherapy source of claim 1, wherein the flexible core comprises a gold strand.

3. The radiotherapy source of claim 1, wherein the flexible core comprises a polymer which is impermeable to radon.

4. The radiotherapy source of claim 3, wherein the flexible core comprises Polyether ether ketone (PEEK).

5. The radiotherapy source of claim 3, wherein the flexible core has a thickness not greater than 0.3 millimeters.

6. The radiotherapy source of claim 1, wherein the radium radionuclides are dispersed throughout a thickness of the polymer coating.

7. The radiotherapy source of claim 1, further comprising small particles dispersed in the polymer coating, wherein the radium radionuclides are coupled to the small particles.

8. The radiotherapy source of claim 1, wherein the flexible core and the polymer coating are not biodegradable for at least a week from implantation in a tumor.

9. The radiotherapy source of claim 1, further comprising a layer of manganese oxide on the flexible core, and wherein the radium radionuclides are coupled to the layer of manganese oxide.

10. The radiotherapy source of claim 9, further comprising a layer of parylene or silicone rubber, between the flexible core and the layer of manganese oxide.

11. A method for preparing a radiotherapy source, comprising:
mixing a solvent and solute to form a mixture which upon curing forms a polymer;
mixing alpha-emitting radium radionuclides into the mixture;
placing the mixture of radium and polymer components on a flexible core; and
allowing the mixture to cure into a polymer coating, after placing the mixture on the flexible core.

12. The method of claim 11, wherein mixing alpha-emitting radium radionuclides into the mixture comprises mixing a solution including radium radionuclides into the mixture.

13. The method of claim 12, further comprising removing excess liquid from the mixture, before placing the mixture on the flexible core.

14. A medicament for treating a tumor, comprising:
microparticles having an outer surface comprising a manganese oxide; and
alpha-emitter radium radionuclides on the outer surface of the microparticles.

15. The medicament as in claim 14, wherein the microparticles comprise a non-manganese-oxide core coated by manganese oxide.

16. The medicament as in claim 14, wherein the microparticles comprise gold, titanium, titanium oxide, zirconium oxide and/or silicon oxide.

17. The medicament as in claim 14, wherein the microparticles have a diameter of less than 10 micrometers.
